(19) United States
(12) Patent Application Publication
(10) Pub. No.: US 2003/0059847 A1
(43) Pub. Date: Mar. 27, 2003

(54) Combinatorial protease substrate libraries

(75) Inventors: Bradley J. Backes, Chicago, IL (US); Jennifer Leslie Harris, San Diego, CA (US)

Correspondence Address: QUINE INTELLECTUAL PROPERTY LAW GROUP, P.C., P O BOX 458, ALAMEDA, CA, 94501, US

(73) Assignee: IRM, LLC, Hamilton (BM)

(21) Appl. No.: 10/229,950

(22) Filed: Aug. 27, 2002

Related U.S. Application Data
(60) Provisional application No. 60/315,116, filed on Aug. 27, 2001.

(51) Int. Cl. G01N 33/53; C12Q 1/37; C07K 7/06; C07K 7/08

(52) U.S. Cl. 435/7.1; 435/23; 530/324

(57) ABSTRACT

Non-peptide protease substrate libraries and high purity protease substrate libraries are constructed, e.g., using fluorogenic compounds. The libraries are useful in obtaining substrate profiles for a variety of proteases, such as methods for determining both prime and non-prime protease recognition sequences.

### CROSS REFERENCE TO RELATED APPLICATIONS

Pursuant to 35 U.S.C. §§119, 120, and any other applicable statute or rule, the present application claims benefit of and priority to U.S. Patent Application Serial No. 60/315,116, filed Aug. 27, 2001, entitled “Combinatorial Protease Substrate Libraries,” the disclosures of which is incorporated herein by reference in its entirety for all purposes.

### COPYRIGHT NOTIFICATION

Pursuant to 37 C.F.R. 1.71(e), a portion of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

The substrate specificity of an enzyme is an important characteristic that typically governs its biological activity. Characterization of substrate specificity provides invaluable information for a complete understanding of complex biological pathways. In addition, understanding of substrate specificity provides a basis for design of selective enzymatic substrates and inhibitors.

Proteases are an important family of enzymes that is crucial to every aspect of an organism's life. In fact, proteases make up at least 2% of the gene products of known genomes. In addition, new proteases are still being identified. New methods are desired to more rapidly assess the substrate specificity of proteases. While several methods are presently used, none are available to rapidly and continuously monitor proteolytic activity against complex mixtures of substrates in solution.

For example, substrate specificity can be probed using peptides displayed on filamentous phage (See, e.g., Matthews and Wells (1993) Science 260, 1113-1117), using combinatorial libraries (See, e.g., Lam and Lebl (1998) Methods Mol. Biol., 87, 1-6), or using 7-amino-4-methylcoumarin fluorogenic peptide substrates (See, e.g., Zimmerman et al. (1977), Anal. Biochem. 78, 47-51). However, none of these methods offer complete and rapid characterization of substrate specificity.

New or improved methods of providing libraries and screening them for substrate specificity are accordingly desirable. The present invention fulfills these and other needs that will become apparent upon complete review of this disclosure.

### SUMMARY OF THE INVENTION

The present invention provides improved protease substrate libraries, and methods of characterizing these libraries to provide complete substrate specificity profiles. For example, the invention provides high purity enzyme substrate libraries for analysis of substrate specificity. The libraries can use positional scanning techniques, for example. Methods of making such libraries are also provided. In addition, the invention provides methods of making non-peptide substrate libraries. Furthermore, methods of obtaining complete substrate specificity profiles are provided.

In one aspect, the present invention provides high purity substrate libraries and methods of preparing such libraries. These methods of preparing one or more fluorophore-containing enzyme substrates typically involve: a) coupling one or more fluorogenic compounds to a solid support via an ammonia-cleavable linker, resulting in one or more support-bound fluorogenic compounds; b) coupling one or more substrate moieties to the support-bound fluorogenic compound; and c) exposing the support-bound fluorogenic compound to ammonia, thereby releasing the fluorogenic compound from the support, resulting in a fluorophore-containing enzyme substrate. A variety of fluorogenic compounds can be used, including coumarin compounds such as 7-amino-4carbamoylmethylcoumarin, 7-amino-4-methylcoumarin, and the like.

The enzyme substrates that comprise the library are often substantially free of, for example, protecting groups that were used in the synthesis methods. In previously available synthesis methods, protecting groups were typically cleaved from the substrates under the same conditions as are used to release the enzyme substrates from a solid support upon which the enzyme substrates were synthesized. The present invention allows removal of the protecting groups prior to release of the enzyme substrates from the solid support, thereby facilitating purification of the enzyme substrates from the removed protecting groups.

One or more substrate moieties are then coupled to the one or more support bound coumarins. If a protected coumarin is used, the substrate moiety is coupled after deprotection of the protected coumarin compound. The substrate moieties provide a putative recognition site for the enzyme of interest. Useful substrate moieties include, but are not limited to amino acids, peptides, non-peptides, and the like. To facilitate synthesis, the substrate moieties can be protected using a suitable protecting group, such as Fmoc. For example, amino acids used as substrate moieties Fmoc protected amino acids, e.g., for performing Fmoc-based peptide synthesis using the support bound coumarin as a starting point.

Fmoc-based peptide synthesis typically comprises coupling a first Fmoc amino acid to the support bound coumarin, resulting in a bound Fmoc amino acid; and deprotecting the bound Fmoc amino acid, resulting in a first bound amino acid. These steps are repeated to produce a desired number of bound amino acids, e.g., about 1 to about 10 amino acids in the present invention. After the desired number of residues is added to the support bound coumarin to form an elongated substrate, protecting groups on the amino acid side chains are removed, e.g., using acid deprotection. When an acid labile linker is used to attach the coumarin compound to the support, it is also cleaved in this step. However, the present invention typically makes use of a linker that is stable to the acid deprotection step used to remove side chain protecting groups. Therefore, the deprotection step does not cleave the substrate from the solid support.

The fluorophore-containing substrate is then exposed to ammonia, e.g., gaseous ammonia mixed with tetrahydrofuran, thereby releasing the fluorogenic compound from the support, resulting in an unbound fluorophore-containing substrate, such as a coumarin-based protease substrates.

In another aspect, the present invention provides fluorophore-containing substrate libraries, such as positional scanning libraries for profiling protease substrate specificity. The libraries are typically produced using the above methods. These libraries are high purity libraries in that the libraries are substantially free of side products, such as protecting group derived side products. Such libraries typically comprise at least about 10, at least about 100, or at least about 1000 members. In some embodiments, the libraries can include 10,000 members or more, greater than about 50,000 members, or greater than about 100,000 members.

In another aspect, the present invention provides non-peptide substrate libraries and methods of making and identifying non-peptide substrates. Methods of making non-peptide substrates typically comprise providing a support bound fluorogenic compound, e.g., a coumarin compound, and coupling an amino acid to the support bound fluorogenic compound. One or more non-peptide molecules are then coupled to the amino acid, e.g., using solid phase synthesis, to form a putative non-peptide protease substrate. For example, a non-peptide substrate is optionally constructed by forming a heterocycle moiety on the amino acid or using benzodiazepine solid phase synthesis. The putative substrate, e.g., removed from the solid support, is then typically contacted with a protease to determine whether the protease cleaves the putative substrate.

Methods of identifying one or more non-peptide substrates for a protease, are also provided. For example, a putative protease substrate is provided that includes a fluorogenic compound, one or more amino acids attached to the fluorogenic compound, and one or more non-peptide molecules attached to the amino acid, such as those made using the methods described above. The putative substrate or a library of such is then contacted with a protease. The method further comprises determining whether the protease cleaves the putative protease substrate, e.g., by detecting a shift in the excitation and/or emission maxima of the fluorogenic compound, which shift results from cleavage of the fluorogenic compound from the amino acid.

In another aspect, the present invention provide libraries of non-peptide protease substrates made by the above methods. These protease substrates typically include a fluorogenic compound, such as a coumarin compound. Proteases of interest include, but are not limited to a serine protease, a threonine protease, a metalloprotease, a cysteine protease, or an aspartyl protease, e.g., caspase, thrombin, plasmin, factor Xa, tissue plasminogen activator, trypsin, chymotrypsin, elastase, papain, or cruzain, and the like.

In another aspect, the present invention provides methods of obtaining a substrate profile for a protease. The methods typically comprise providing a library of putative protease substrates, each of which comprises a putative protease recognition site, and incubating the library in the presence of the protease. Typically the library is formed to provide a positional scanning combinatorial library. The cleavage reactions are then monitored, thereby providing the substrate profile for the protease.

The putative protease recognition site typically comprises one or more nonprime positions and one or more prime positions, each of which positions is occupied by a substrate moiety. The prime and non-prime positions flank a putative protease cleavage site, with the non-prime positions being defined as being on the amino-terminal side of the cleavage site, and the prime positions being on the carboxy-terminal side of the cleavage site. The substrate moieties that occupy the non-prime positions are preselected to allow cleavage of the substrate at the putative protease cleavage site by the protease; and the substrate moieties that occupy the prime positions vary among different members of the library of protease substrates.

The substrate moieties that occupy one or more of the non-prime positions are typically preselected by providing a first library comprising one or more putative protease substrates, each of which comprises a fluorogenic compound and a putative protease recognition site. The putative protease recognition site is flanked by a putative protease cleavage site and comprises one or more non-prime positions, each of which positions is occupied by a substrate moiety. This library is incubated in the presence of the protease of interest and library members that are cleaved by the protease are identified, thereby identifying substrate moieties that, when present in a particular non-prime position, allow cleavage of the substrate by the protease at the putative protease cleavage site. Cleavage of the members of this library is determined by detecting a shift in the excitation and/or emission maxima of the fluorogenic compound, which shift results from release of the fluorogenic compound from the putative protease recognition site. The substrate moieties identified are then used to construct a prime side scan as described herein.

Cleavage of the protease substrate compounds in the prime side scan is typically detected by fluorescence resonance energy transfer, in which case, a donor and an acceptor moiety are attached to the protease substrate compound on opposite sides of the putative protease cleavage site.

The methods described above, also optionally comprise determining one or more kinetic constants cleavage of the substrate, e.g., by detecting release of the fluorogenic compound. Kinetic data is typically obtained by detecting the fluorogenic compound at multiple time points in the course of the cleavage reaction. This data and the data regarding the preferred substrates are optionally used in databases as described below.

In another aspect, the present invention provides databases of substrate profile information for a protease or for a plurality of proteases, wherein the database comprises records for members of a library of putative protease substrates. Each record typically comprises information as to the identity of a substrate moiety or group of substrate moieties that occupy each of one or more prime and non-prime positions of the particular putative protease substrate, as well as data from assays to determine the ability of the protease or proteases to cleave the putative protease substrate. The information for each record is typically obtained using the methods described herein. Kinetic information obtained at multiple time points is also optionally included in the databases.

### BRIEF DESCRIPTION OF THE FIGURES

FIG. 1 provides a traditional scheme to prepare 7-amino-4carbomoylcoumarin (ACC) substrate libraries.

FIG. 2 provides a plan for preparing a non-prime side scan for substrate specificity.

FIG. 3 illustrates gaseous cleavage of coumarin-based substrate libraries from a solid support.

FIG. 4 illustrates preparation of a coumarin-based substrate of the invention on a solid support.

FIG. 5 provides one example of a pathway for preparation of a non-peptide-based substrate.

FIG. 6 provides a second example of a pathway for preparation of a non-peptide substrate.

FIG. 7 shows results of a thrombin non-prime scan for substrate specificity.

FIG. 8 illustrates an example substrate for a prime-side scan for substrate specificity.

FIG. 9 illustrates a variety of donor and acceptor moieties, e.g., fluorescence resonance energy transfer pairs, for use in a prime side scan for substrate specificity.

FIG. 10 shows results for a prime scan for thrombin using an optimal non-prime sequence of P1-arg, P2-pro, P3-variable, P4-aliphatic or aromatic amino residue.

FIGS. 11A and 11B show a 4 (1H)-Quinazolinone, 6-chloro-2-(5-chloro-2-hydroxy-phenyl)-2,3-dihydro-(9C1), which is suitable for use as a fluorogenic compound in the methods and libraries of the invention. FIG. 11A shows the quinazolinone compound in the absence of attached amino acids. FIG. 11B shows the quinazolinone compound attached to four amino acids, which represent positions P1 through P4 of a protease recognition site.

### DETAILED DESCRIPTION OF THE INVENTION

The present invention provides libraries and methods for profiling enzymatic substrate specificity, such as for determining recognition sequences for proteases. The substrate specificity of a protease is an important characteristic that often governs its biological activity. Knowledge of substrate specificity can help to, for example, identify macromolecular substrates for a given protease, thus shedding light on its biological activity. Substrate specificity can also guide the design and generation of potent and selective substrates and inhibitors. Therefore, the present invention provides methods and libraries for profiling substrate specificity.

High purity fluorogenic enzyme substrate libraries are provided in one aspect of the invention. Methods of making the libraries are also provided. As an example, the invention provides high purity coumarin-based libraries, including peptide and non-peptide libraries. The high purity libraries provide for rapid analysis of large substrate libraries without a prior purification step and with greater sensitivity due to the high purity of library.

The protease substrate libraries of the invention are useful in obtaining a complete substrate profile of a protease. For example, positional scanning techniques can be employed using the methods and libraries of the invention. The invention provides novel libraries and methods of creating them, as well as novel methods of profiling enzymes. For example, a novel profiling method is provided for determining optimal substrate sequences on either side of a cleavage site.

In another aspect, methods of making non-peptide substrate libraries, e.g., coumarin-based non-peptide substrate libraries, are provided. These libraries are used, e.g., to identify novel protease substrates.

In another aspect, the present invention provides an enzyme profiling method that provides putative substrate sequences for both prime and non-prime sides' of the substrate, e.g., optimal or preferred compositions for each side of the cleavage site.

Definitions

Enzymes are biological catalysts that typically catalyze chemical reactions in living cells. Typical enzymes comprise proteins or nucleic acid molecules, e.g., RNA. Substrates are the recipients of enzymatic catalysis. For example, a proteolytic enzyme acts upon a protein or peptide substrate by hydrolyzing one or more peptide bond.

The terms “polypeptide,” “peptide,” and “protein” are used interchangeably herein to refer to a polymer of amino acids linked through peptide bonds. Polypeptides of the invention include, but are not limited to, proteins, biotinylated proteins, isolated proteins, recombinant proteins, enzymes, enzyme substrates and the like. In addition, the polypeptides or proteins of the invention optionally include naturally occurring amino acids as well as amino acid analogs and/or mimetics of naturally occurring amino acids, e.g., that function in a manner similar to naturally occurring amino acids. In the present invention, amino acids are typically used to create peptides and proteins for positional scan substrate libraries. The positional scan libraries are used to determine optimal substrate sequences for enzymes, e.g., proteolytic enzymes.

A typical enzyme of interest in the present invention is a protease. “Protease,” as used herein, typically refers to an enzyme that degrades proteins or peptides by hydrolyzing peptide bonds between amino acid residues. In some embodiments, proteases, also known as proteinases, peptidases, or proteolytic enzymes, are used to cleave non-peptide substrates. Various types of proteases are optionally studied using the libraries and methods of the present invention, including, but not limited to serine proteases, threonine proteases, metalloproteases, cysteine proteases, aspartyl proteases, and the like. Example proteases include, but are not limited to, carboxypeptidase A, subtilisin, papain, pepsin, thrombin, plasmin, factor Xa, tissue plasminogen activator, caspase, trypsin, chymotrypsin, elastase, cruzain, and the like.

Many proteases are non-specific in their activity, meaning that they digest proteins to peptides and/or amino acids. Other proteases are more specific, cleaving only a particular protein or only between certain predetermined amino acids. Still other proteases have optimal sequences that they cleave preferentially over others. The methods and substrates of the present invention are used to screen protease substrates to determine optimal peptide sequences that a given protease will recognize and cleave. In addition, the present invention provides non-peptide substrates that are used to identify novel sequences cleavable by a protease of interest.

“Protease substrates” of the present invention include, but are not limited to, proteins, polypeptides, peptides, and the like. A protease catalyzes the hydrolysis of a protease substrate, e.g., a protein or polypeptide, producing degraded protein products. In the present invention, protease substrates also include non-peptide substrates. For example, a coumarin-based substrate comprising an amino acid and a non-peptide moiety optionally serves as a protease substrate. Such novel substrates are optionally used to further explore the specificity of proteases.

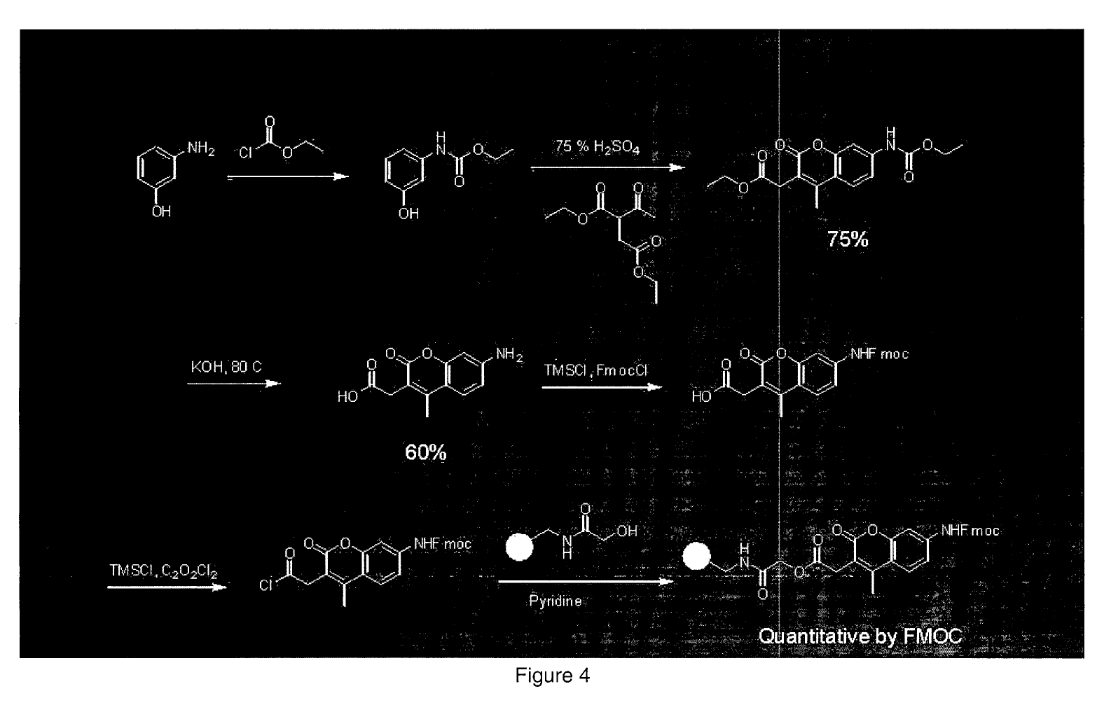

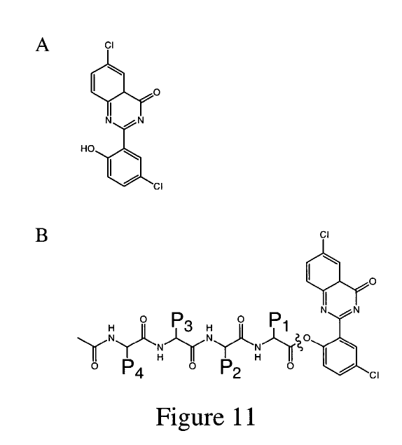

Typically, the substrates of the present invention include a fluorogenic compound. When a protease cleaves the substrate, a detectable change in fluorescence typically occurs. Examples of suitable substrates Are “coumarin based substrates,” which are substrates that include coumarin and one or more substrate moieties, such as amino acids. Coumarin compounds of interest in the present invention include, but are not limited to, 7-amino-4-carbamoylmethylcoumarin (ACC), 7-amino-4-methylcoumarin (AMC), and 7-amino-3-carbamoylmethyl-4-methylcoumarin, and the like. The synthesis of an example coumarin compound of interest is shown in FIG. 4. Amino-phenol is acylated, e.g., with ethylchloroformate, to provide a carbamate. The carbamate is reacted with diethylacetyl succinate, e.g., in the presence of sulfuric acid, to provide a diprotected coumarin compound. The protecting groups on the coumarin are removed, e.g., using potassium hydroxide, to provide a free coumarin, such as aniline coumarin. Many other coumarin compounds are available, either commercially (See, e.g., Sigma and Molecular Probes catalogs) or using various synthetic protocols known to those of skill in the art. Another example of a suitable fluorogenic compound is 4 (1H)-Quinazolinone, 6-chloro-2-(5-chloro-2-hydroxy-phenyl)-2,3-dihydro-(9C1) (FIG. 11). See, e.g., Naleway, J J; Fox, C M J; Robinhold, D; Terperschnig, E; Olson, N A; Haugland, R P. (1994) “Synthesis and use of new fluorogenic precipitating substrates.” Tet Letters 35 (46): 8569-8572.

A “substrate moiety” is any amino acid, peptide, protein, non-peptide moiety, small molecule, organic molecules, inorganic moiety, or the like that can be coupled to a fluorogenic compound, such as a coumarin compound. Typically, the non-peptide, amino acid, or peptide used as a substrate moiety forms an amide linkage with a fluorogenic compound and leaves a carbonyl linkage available for further coupling reactions. Once coupled to a fluorogenic compound, for example via an amide bond, a substrate moiety becomes part of a fluorophore-containing substrate that is used as a protease substrate. The compounds can then be used to probe substrate specificity.

I. Preparation of High Purity Fluorophore-Based Substrates

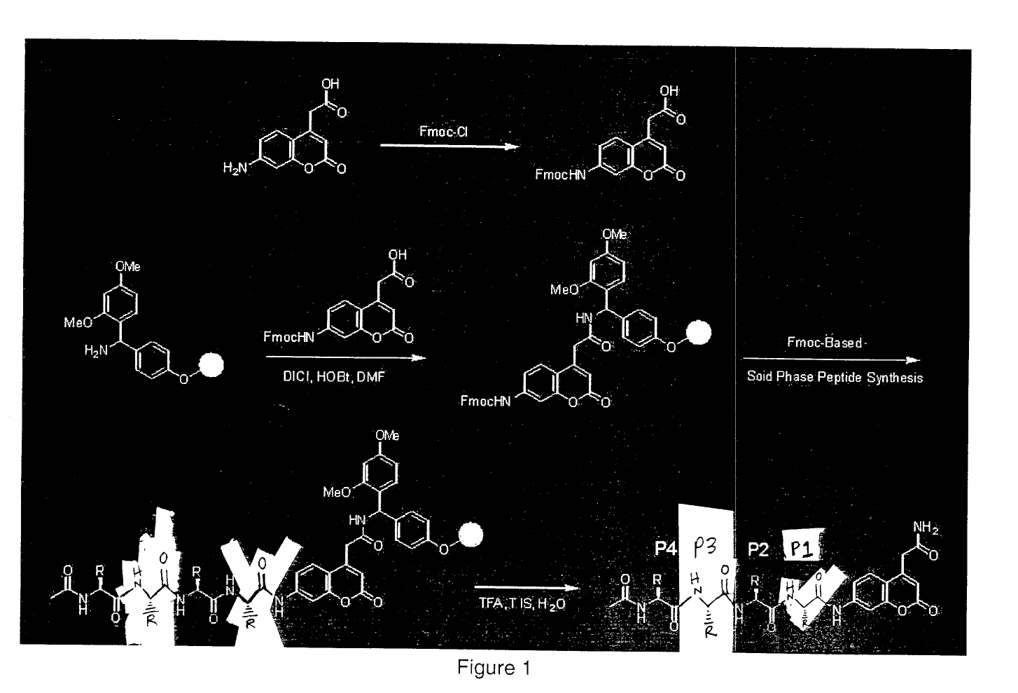

The present invention provides a strategy for the preparation of high purity libraries of fluorogenic substrates, including coumarin-based substrates. In traditional solid-phase methods of fluorophore-based substrate library production (See, e.g., FIG. 1), the resulting substrates are mixed with side chain protecting group side products because the substrate is cleaved from the support at the same time as the side chains are deprotected by, for example, using trifluoroacetic acid (TFA) and triisopropylsilane (TIS). When preparing libraries that consist of multiple wells with multiple substrates in each well, it is very difficult to purify all wells, and often the residual impurities from the protecting groups employed in the synthesis deactivate a sensitive protease. The present invention solves this problem by providing high purity substrate libraries that do not contain side chain deprotecting group side products.

By using the linker strategy described herein, solid phase strategies are possible in which protecting groups are cleaved without removal of the substrates from the resin, thereby avoiding contamination of the substrate library with side products such as protecting group derived side products. Protecting group side products can be washed away, after which a discrete cleavage step is used to remove compounds from the resin. With this strategy, pure libraries are optionally established for use with a wide range of proteases.

For basic strategies for preparation of and use of coumarin-based libraries, see, e.g., Zimmerman, M., Ashe, B., Yurewicz, E. & Patel, G. (1977) Analytical Biochemistry 78, 47-51; Lee, D., Adams, J. L., Brandt, M., DeWolf, W. E., Jr., Keller, P. M. & Levy, M. A. (1999) Bioorganic and Medicinal Chemistry Letters 9, 1667-72; Rano, T. A., Timkey, T., Peterson, E. P., Rotonda, J., Nicholson, D. W., Becker, J. W., Chapman, K. T. & Thornberry, N. A. (1997) Chemistry and Biology 4, 149-55; Schechter, I., Berger, A. (1968) Biochemical and Biophysical Chemistry Communications 27, 157162; Backes, B. J., Harris, J. L., Leonetti, F., Craik, C. S. & Ellman, J. A. (2000) Nature Biotechnology 18, 187-193; Harris, J. L., Backes, B. J., Leonetti, F., Mahrus, S., Ellman, J. A. & Craik, C. S. (2000) Rapid and general profiling of protease specificity by using combinatorial fluorogenic substrate libraries, Proc Natl Acad Sci USA. 97, 7754-7759. See, also, Smith et al. (1980) Thrombosis Research 17, 393-402.

Coupling Afluorophore Compound to a Solid Support.

To prepare a fluorophore-based enzyme substrate, a fluorogenic compound is attached to a solid support via a linker molecule. For example, the fluorogenic compound can be a coumarin compound, e.g., 7-amino-4-carbamoylmethylcoumarin (ACC), 7-amino-4-methylcoumarin (AMC), 7-amino-3-carbamoylmethyl-4methylcoumarin, or the like. Typical solid supports comprise resins or polymers, such as polymer beads. Polystyrene, polyethylene, polypropylene, polyethylene glycol, polyacrylamide, or the like are examples of materials that can be used to provide a solid support. For example, a plurality of polystyrene beads in a plurality of microwells is optionally used to provide a solid support of the invention. A fluorogenic compound is typically coupled to the solid support, e.g., attached or bonded, through a linker molecule, to provide a support-bound fluorogenic molecule.

The linker molecules used in the methods and libraries of the invention are preferably ammonia-labile. Such linkers include, for example, glycol linkers and benzylalcohol linkers. In traditional protocols, the linker used to prepare a fluorophore-based substrate is an acid labile linker that is cleaved in an acid deprotection step used to remove protecting groups from the amino acid side chains. However, in the present invention, the linker group is typically an ammonia-labile linker group that allows the fluorophore-based substrate to remain coupled to the solid support even when subsequent acid deprotection is used to deprotect various side chains. One example of a suitable linker is the glycol linker as shown in FIG. 4.

The linker used in the methods of the invention is also stable to conditions used to cleave other protecting groups that are used in solid-phase synthesis. For example, to aid in synthesis of the substrate libraries, the fluorogenic compounds and amino acids or other substrate moieties that are attached to the fluorogenic compounds can be protected by, for example, 9-fluorenylmethoxycarbonyl (Fmoc). FIG. 4 shows a free coumarin that is mono-protected using an Fmoc protecting group as used in typical Fmoc peptide synthesis protocols. In the example shown in FIG. 4, the a-amino group is protected prior to coupling to the solid support, e.g., using a 9-fluorenylmethoxycarbonyl (Fmoc) protecting group on the coumarin amino group. This preserves the α-amino group from reaction prior to coupling to a substrate moiety. The Fmoc-protected coumarin is then used to prepare an acid-chloride coumarin which is coupled to a solid support via a glycol linker (shown) or a benzylalcohol linker. The protecting group is typically removed prior to the next step, which is typically coupling of the substrate moieties to the support-bound fluorogenic compound.

Coupling a Substrate Moiety to a Support Bound Fluorogenic Compound

Once a fluorogenic compound is attached to a solid support, a substrate moiety is coupled to the fluorogenic compound. A substrate moiety is any molecule, amino acid, peptide, or the like that forms a bond with the fluorogenic compound. For example, the substrate moiety can have a carboxyl group that is used to form an amide or ester bond to the fluorogenic compound, and a free amino group that is used to couple additional substrate moieties. However, for substrate synthesis, e.g., peptide synthesis, the α-amino group of the substrate moiety is protected. Generally, it is preferred to use a base-labile protecting group for this purpose, so that one can remove these protecting groups without simultaneously removing the side chain protecting groups. Fmoc is one example of a suitable base-labile protecting group that can be used during the coupling reaction. The Fmoc group is then removed in a deprotecting reaction and the fluorophore-based substrate is optionally subjected to further elongation with more substrate moieties, such as Fmoc protected amino acids.

For example, an Fmoc-amino acid is optionally coupled to a support bound coumarin via an amide bond. The Fmoc group is then removed under basic conditions, to deprotect the amino group, which is then available for further elongation, e.g., with another Fmoc-amino acid. Fmoc peptide synthesis protocols are well known to those in the art.

In some cases, the substrate moieties, e.g., amino acids, typically comprise side chain protecting groups that to protect the side chains from reaction during the synthesis of the substrate. These side chain protecting groups are also removed in a deprotection step. Since it is desirable to leave these side chain protecting groups attached until all substrate moieties have been attached, the side chain protecting groups are typically chosen so that they are not removed by conditions that remove the protecting groups on, for example, the a-amino acid. Often, an acid deprotection step is used. Suitable acid-labile protecting groups include, for example, tert-butoxycarbonyl groups (tBoc). After the substrate moiety is elongated to a desired length, e.g., four amino acids long, the side chain protecting groups are removed to prepare the library for use, for example, in a protease assay to determine substrate specificity of proteases.

Releasing the Coumarin-Based Substrate from the Solid Support

Once the substrate moiety or substrate moieties have been added to the support-bound fluorogenic compound, the substrate is released from the support. The fluorophore-containing substrate can then be used in, for example, a profiling analysis. Typically, one or more amino acid residues are coupled to the support-bound fluorogenic compound in the previous step to form a substrate, e.g., a protease substrate. When complete, e.g., when the desired number of residues have been added (often about 1 to about 6 residues), the substrate is released from the support and incubated in the presence of a protease of interest. Proteases typically cleave the amide bond between the first substrate moiety and the fluorogenic compound. Released fluorogenic compound resulting from the cleavage is detected to determine whether or not the substrate of interest was cleaved by the protease of interest.

In traditional protocols, the fluorophore-containing substrate is released from the support in an acid deprotection step that is used to remove various acid-labile protecting groups from the substrate moieties, such as are sometimes present on amino acid residues that were attached to the fluorogenic compound. However, as discussed above, this leads to an impure substrate, one that is mixed with the removed side chain protecting groups. The use of an ammonia-cleavable linker allows one to remove protecting groups from, for example, amino acid side chains, prior to releasing the fluorophore-containing enzyme substrates from the solid support. During peptide synthesis, protecting groups are often attached to amino acid side chains to prevent amino acids from attaching to the nascent peptide via the side chains. A protecting group is also typically attached to each of the substrate moieties (e.g., amino acids) that are being attached to the nascent peptide to prevent attachment of multiple amino acids. The protecting groups used for amino acid side chains generally differ from those used to prevent multiple attachments in the conditions by which the protecting groups are removed, since it the protecting group on the free end of the peptide must be removed at each step of the synthesis, while it is desirable to leave the side chain protecting groups in place until synthesis of the peptide is complete. Therefore, an acid-labile protecting group is typically used for side chain groups, while a base-labile protecting group is used to protect the α-amino group.

If an acid labile linker is used to attach the fluorogenic compound to the solid support, it is typically cleaved during the acid deprotection of the substrate moiety side chain protecting groups. For example, in Fmoc peptide synthesis, after a desired peptide length is reached, the amino acid side chain protecting groups are removed in an acid deprotection step. The fluorogenic compound is simultaneously cleaved from the solid support if an acid labile linker is used to bind the fluorogenic compound to the solid support. However, this simultaneous cleavage does not provide a very pure library. For example, various side chains products are included in the library of substrates, which is difficult to purify when multiple substrates, e.g., a library of substrates are being simultaneously prepared in one or more microwell plates.

The present invention provides libraries of high purity, e.g., by making the side chain deprotection step orthogonal to the cleavage of the substrate from the support. In other words, the two events are separated into two steps; the side chains are deprotected without simultaneously cleaving the substrate from the support. The present invention provides an ammonia-labile linker that is not cleaved in the acid deprotection step typically used to remove the side chain protecting groups. In addition, the ammonia-labile linkers of the invention are stable to Fmoc deprotection, such that the substrates remain coupled to the support until after all Fmoc and side chain deprotecting steps have been completed. Using this protocol, the removed side chain protecting groups are optionally washed from the reaction solution, while the substrate remains support bound. This allows preparation of a high purity library when the substrates are cleaved from the support as described below.

The substrate is not cleaved from the support until all deprotection and synthesis have taken place. Any unwanted side products or protecting groups are optionally rinsed from the support bound coumarin substrate. Therefore, when the substrate is cleaved from the solid support, it has a very high level of purity, e.g., it contains substantially no side chain products, such as those derived from removed protecting groups. The substrates produced in this manner are typically at least about 85% pure, more preferably about 95% pure and most preferably, about 99-100% pure.

Cleavage of the support bound substrate from the solid support is typically achieved, e.g., after all desired deprotection steps, using ammonia, e.g., gaseous ammonia. See, e.g., Bray et al. (1991) Tetrahedron Letters, 32 6163-6166. The ammonia is optionally concentrated liquid ammonia or gaseous ammonia. In addition, tetrahydrofuran (THF) is optionally used with the ammonia to effect the cleavage of the substrate from the solid support. This cleaves the substrate from the solid support, at which point it is optionally used in an enzymatic assay.

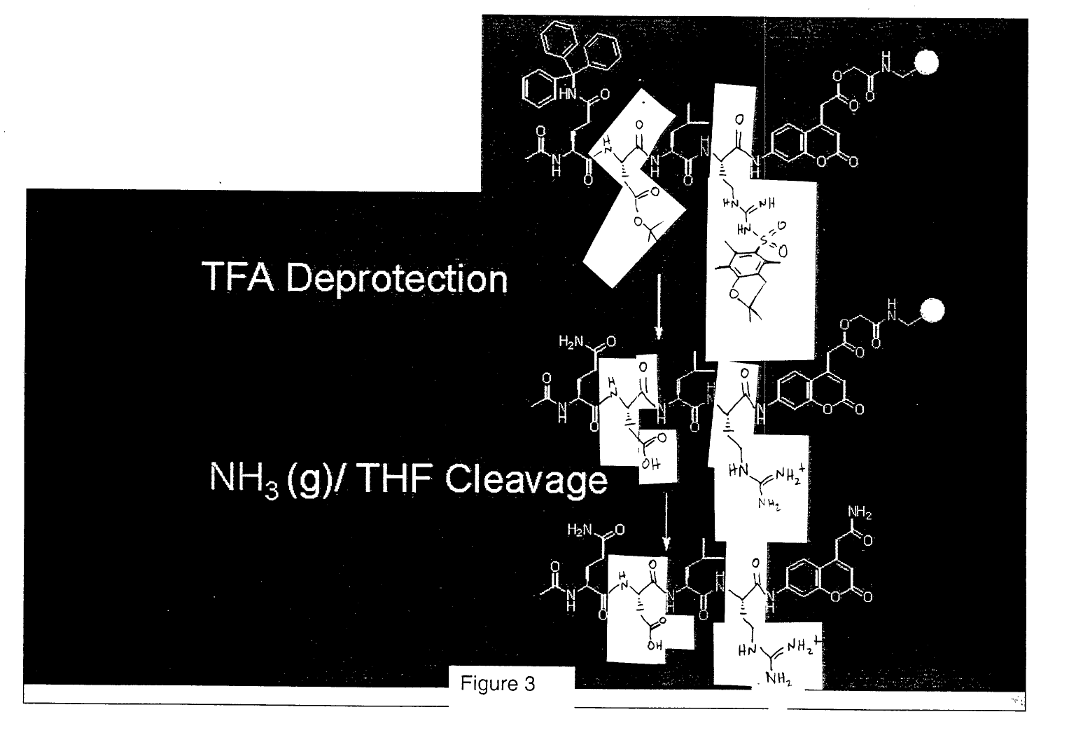

For example, FIG. 3 illustrates a gaseous phase cleavage strategy for use in making a coumarin-based substrate. The coumarin-based substrate in FIG. 3 is optionally prepared as described above. It comprises a glycol linker used to couple 7amino-4-carbamoylmethylcoumarin (ACC) to a solid support, e.g., polystyrene. The substrate moiety coupled to the support bound coumarin comprises four amino acid residues or substrate moieties (P1, P2, P3, and P4) P1 is arginine with a sulfonamide based protecting group on its side chain. P2 is leucine and P3 is aspartic acid with a tert-butyl ester protecting group. P4 is glutamine and a trityl protecting the amide group. Trifluoroacetic acid (TFA) is used to perform an acid deprotection step to remove the protecting groups from the amino acids residues P1-P4. The glycol linker is typically stable to the TFA deprotection. Gaseous ammonia and THF are used to cleave the coumarin-based substrate from the solid support. The released coumarin-based substrate is then available for use, e.g., in an enzymatic assay. The substrate is a high purity substrate as it contains no side products, e.g., protecting group derived side products, because they were removed in an acid deprotection and rinsed away from the solid support, to which the substrate was still bound.

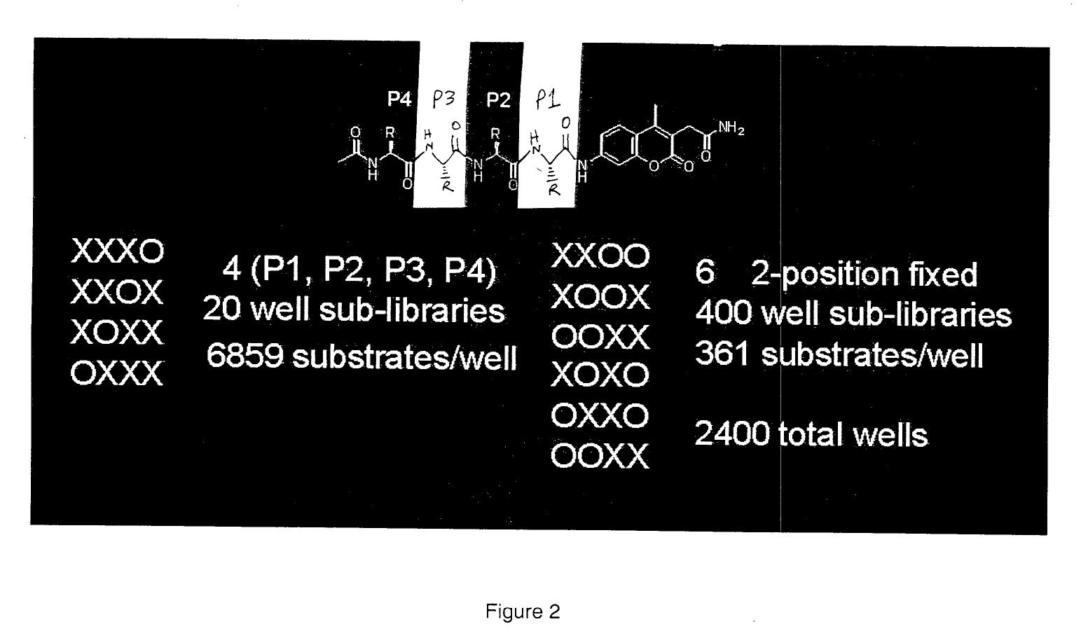

The method described above is particularly useful when making many substrates, e.g., when making a library of fluorescent compound-based substrates. A library of fluorescent compound-based substrates is optionally used as described below to obtain a complete substrate specificity profile of an enzyme. The libraries presented herein, e.g., fluorescent compound-based substrate libraries of high purity, are particularly useful in developing specificity profiles of proteases. A whole library can be created as described above in various microwell plates, as explained in FIG. 2.

FIG. 2 shows a plan to develop a positional scanning library, e.g., for protease substrates. Four 20 well sub-libraries are created, wherein each of the four sub-libraries has a different fixed amino acid position, e.g., P1, P2, P3, or P4. For example, in a first sub-library, each of the twenty wells contains a library of substrates wherein P1 is fixed at one of twenty different amino acids while the other positions, P2, P3, and P4, are varied. (As used herein, the nomenclature for substrates includes prime side and nonprime side positions, Pn, . . . P4, P3, P2, P1, P1′, P2′, P3′, P4′ . . . Pn′, wherein cleavage, e.g., amide bond hydrolysis, occurs between P1 and P1′). See, e.g., Schechter and Berger (1968) Biochem. Biophys, Res. Commun. 27, 157-62.) This produces about 8000 different substrates per well.

Additional sub-libraries are also optionally created, e.g., with two fixed positions, e.g., P3 and P4. This produces six sub-libraries of 400 wells each, wherein each well contains about 400 different substrate sequences. Therefore, the libraries of the invention typically involve about 2400 wells total and the libraries contain well over 100,000 different substrates, e.g., coumarin-based substrates. The preferred amino acid for each position is optionally determined using these positional scanning libraries. See, e.g., Harris et al. (2000) PNAS 97, 7754-7759, for a description of how such libraries are used to determine optimal substrate sequences.

The libraries are created using peptide synthesis techniques well known to those of skill in the art, or the techniques described above to produce high purity libraries. For the varied positions, a mixture of amino acids is added to the coupling reaction to couple a random substrate moiety or amino acid to the support bound coumarin. In addition, the libraries are optionally created using non-peptide molecules in the P1, P2, P3, and/or P4 positions, as described in more detail below.

In another aspect, the present invention provides libraries of substrates, e.g., fluorophore-based libraries, made by the methods described above. These libraries are optionally used to provide non-prime side information regarding the various substrates of the library. For example, a non-prime substrate sequence, e.g., the first four amino acids on the non-prime side of the cleavage site, may be identified as optimal for a particular protease of interest. This information is then optionally used to design more selective and potent substrates. For example, different fluorogenic compounds are optionally employed to increase the sensitivity of these substrates. The substrates identified also provide valuable diagnostics for the identification of protease activity in complex biological samples and are valuable in screening efforts to identify protease inhibitors. For example, the optimal non-prime information is optionally used to design more selective and potent inhibitors, e.g., inhibitors that serve as therapeutic agents or biological tools, to bias the generation of libraries aimed at identifying prime side specificity determinants, and/or provide panning information that allows for the generation of specific substrates and inhibitors in the context of an entire set of proteases. This provides a genomic approach rather than a target-based approach.

In addition, non-peptide substrates rather than peptide-based substrates are optionally prepared employing the above deprotecting and cleavage strategies, e.g., to provide more selective substrates and/or substrates with improved pharmacokinetic profiles than peptide based substrates.

II. Preparation of Non-Peptide Substrates

The libraries and methods presented herein are typically used to identify the substrate specificity of proteases. For example, the libraries include positional scanning libraries of fluorogenic peptide substrates in which a tremendous amount of diversity space is represented in a limited number of wells. The fluorogenic signal that proteolysis generates can be monitored continuously with great sensitivity to reveal the substrate specificity of a protease of interest. Knowledge of the substrate specificity for a collection of proteases is optionally used to guide the design and generation of potent and selective substrates and inhibitors. The ability to synthesize libraries of non-peptidic substrates for assay with proteases is valuable in the identification of more selective and potent substrates because unexplored areas of the protease binding pocket may be accessed. For in vivo applications, non-peptide substrates also demonstrate better pharmacokinetic properties than peptidic substrates. For instances in which the optimal substrate identified is engineered to provide inhibitors, e.g., by substituting the scissile peptide bond with a protease-class specific warhead, non-peptide inhibitors, e.g., small molecule inhibitors, are more likely than peptide-based inhibitors to have drug-like properties. Therefore, the present invention provides methods of making non-peptide protease substrates.

These non-peptide substrates are optionally prepared employing the above strategies, such as gas phase cleavage of a substrate from a solid support. Alternatively, more traditional strategies are also optionally used, including those in which protecting groups, if necessary for the non-peptide substrate moieties, are cleaved simultaneously with cleavage from the support.

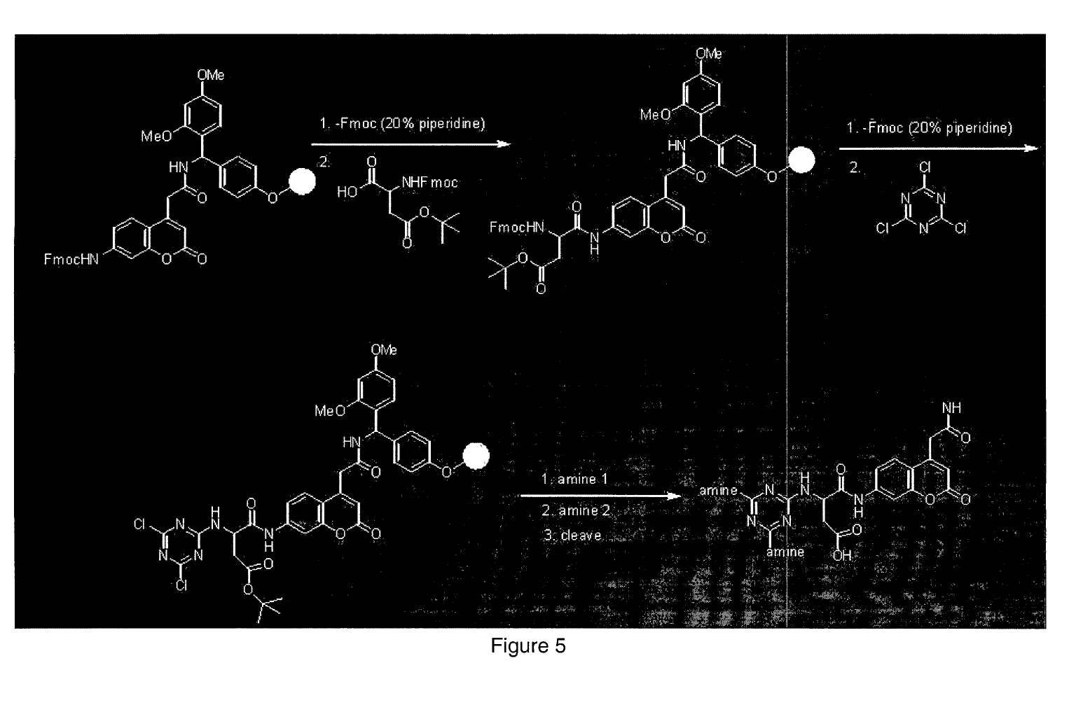

Using a support-bound fluorogenic compound, e.g., a coumarin compound, non-peptide libraries are optionally constructed employing a fixed P1 amino acid, e.g., to focus the library on proteases that have a significant P1 preference. For example, aspartic acid is optionally positioned to provide a library that is focused for use with caspase. See, e.g., FIG. 5, in which a heterocycle is constructed on the amino terminus of the P1 amino acid employing standard solid-phase synthesis strategies. Libraries constructed in this manner optionally provide new substrates that access new portions of the binding pocket of the protease, e.g., portions of the binding pocket that presently available peptide backbones can not exploit.

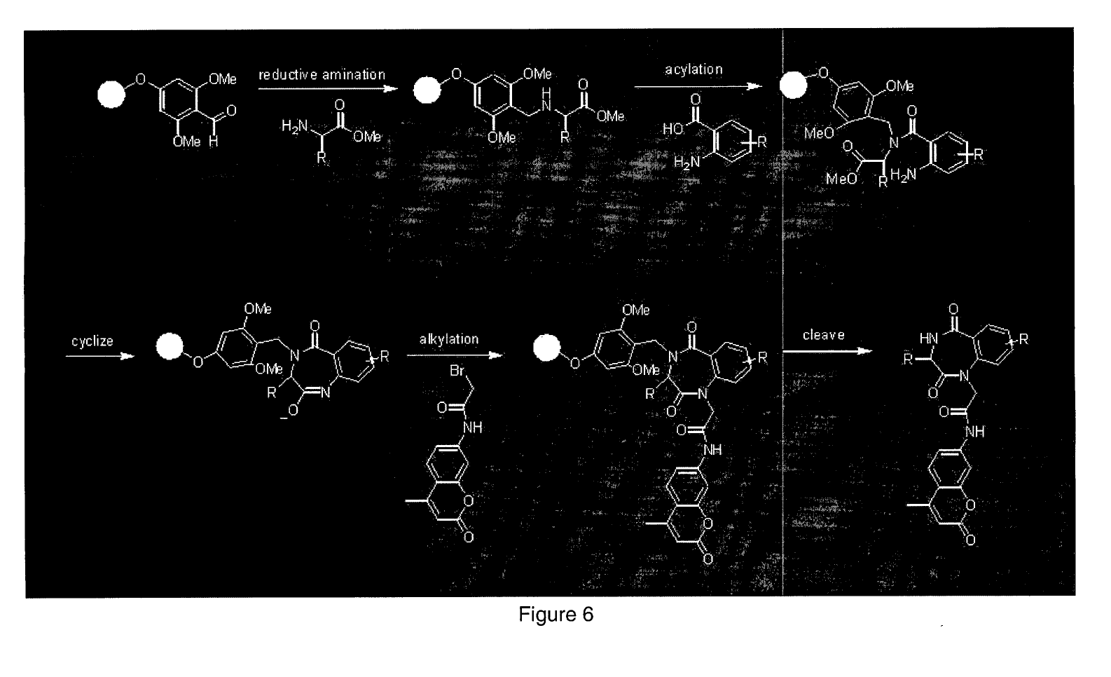

It is also possible to prepare non-peptide substrates on a large number of non-peptidic scaffolds by incorporating reactive coumarin-containing building blocks. For example, FIG. 6 illustrates a classic benzodiazepine solid-phase strategy used to construct non-peptide substrates by using a coumarin-containing alkylating agent. By employing positional scanning methods, a tremendous amount of substrate space is optionally covered in a limited number of wells. Non-peptidic substrates may also be more selective, and provide better starting points for the design of inhibitors with good pharmacokinetic properties.

In one aspect, a method of identifying one or more non-peptide substrates for a protease, is provided. The method typically comprises providing a support bound fluorogenic compound, e.g., a coumarin compound, and coupling one or more amino acids to the support bound fluorogenic compound. The amino acids are chosen to provide a preferred cleavage site, adjacent to the first non-prime position, P1. Fluorogenic compounds of interest include coumarin compounds such as, 7-amino-3carbamoylmethyl-4-methylcoumarin; 7-dimethylamino-4-carbamoylmethylcoumarin, 7amino-4-carbamoylmethylcoumarin, and 7-amino-4-methylcoumarin, and the like.

One or more non-peptide molecules are then coupled to the P1 amino acid to form a putative non-peptide protease substrate. A “putative substrate” as used herein refers to a supposed substrate molecule, e.g., one that typically has not been tested, yet but is supposed to act or is assumed to act as a substrate for one or more enzyme. Typical non-peptide molecules used as substrate moieties in the present invention include, but are not limited to alkyls, aryls, phenyl and benzyl compounds, phenols, alcohols, alkynes, methyl, ethyl, propyl, isopropyl, butyl, tert0butyl, cyclohexyl, other small organic molecules, and the like.

The putative non-peptide protease substrate is then contacted with a protease to determine whether the protease cleaves the putative substrate. Typically, the putative substrate is removed from the solid support prior to reacting with the enzyme of interest, e.g., using gaseous ammonia as described above or traditional methods involving acidic cleavage of an acid labile linker.

Typically, standard solid phase synthesis methods are used to couple the amino acid to the fluorogenic compound and to couple the one or more non-peptide moieties to the amino acid. Standard peptide synthesis methods are optionally used to couple the amino acid. Other standard protocols exist and are well known to those of skill in the art to perform solid phase synthesis of the type used here. See, e.g., Backes and Ellman, J. Org. Chem. (1999) 64, 2322-2330; and Thompson and Ellman, (1996) Chem Rev. 96 555-600, and the references cited therein.

Two example methods of coupling non-peptides to the amino acid to form non-peptide substrates are illustrated in FIGS. 5 and 6. FIG. 5 illustrates an Fmoc-protected coumarin compound coupled to a solid support via a Rink linker. The Fmoc group is removed from the coumarin compound, e.g., in piperidine, and an aspartic acid is coupled to the coumarin. The bound amino acid is then reacted with trichlorotriazine, e.g., in a S N-aryl substitution reaction, to provide a support bound heterocycle that is optionally selectively substituted with amines. In this manner, a non-peptide substrate is provided which is biased to proteases that prefer an aspartic acid at the P1 cleavage position.

FIG. 6 provides an example of benzodiazepene solid phase synthesis. See, e.g., Boojamra et al. J. Org. Chem. (1995) 60, 5742-5743. In the final alkylation step coumarin is used to alkylate nitrogen to give a coumarin substituted benzodiazepene. These syntheses are optionally used, e.g., with coumarin building blocks to provide libraries of putative protease substrates that can be analyzed as provided below to identify novel protease substrates or using methods known to those in the art to identify preferred substrates for a protease of interest.

The present invention also provides a library of non-peptide substrates, e.g., made by the methods described above, for analysis as described below. For example, a library of fluorophore-based non-peptidic protease substrates is optionally provided. The amino acid used to provide the P1 position in the putative substrates is optionally any amino acid, e.g., to bias the library to provide substrates for one or more protease, e.g., a serine protease, a thiol protease, a metalloprotease, a cysteine protease, a carboxyl protease, or the like. Example proteases of the invention, include, but are not limited to, caspase, thrombin, plasmin, factor Xa, tissue plasminogen activator, trypsin, chymotrypsin, elastase, papain, cruzain, and the like.

For example, methods of identifying non-peptide protease substrates are provided. The methods typically comprise providing a putative protease substrate, e.g., as described above. For example, a typical putative substrate of the invention comprises a fluorogenic compound, e.g., a coumarin, an amino acid attached to the fluorogenic compound, and one or more non-peptide molecules attached to the amino acid. The putative protease substrate is then contacted with a protease. The method further comprises determining whether the protease cleaves the putative protease substrate. Detection is typically accomplished by detecting a shift in the excitation and/or emission maxima of the fluorogenic compound, which shift results from cleavage of the fluorogenic compound from the amino acid. Additional methods of profiling substrate libraries are provided below.

III. Obtaining a Complete Substrate Profile of a Proteolytic Enzyme

The present invention also provides methods for rapidly obtaining a complete substrate specificity profile for an enzyme, e.g., for a protease. The substrate specificity of an enzyme is an important characteristic that governs its biological activity. Knowledge of substrate specificity is useful in identification of macromolecular substrates for a given enzyme, thus shedding light on its biological activity. Substrate specificity is also used to guide the design and generation of substrates and inhibitors. The present invention therefore provides a strategy to rapidly obtain complete substrate specificity profiles, e.g., for proteases. By employing libraries of fluorogenic substrates in a positional scanning format, information regarding the non-prime specificity is rapidly obtained in an initial profiling experiment, e.g., as described above and in the references cited therein. The present methods extend this profiling method to include a prime side specificity scan. Therefore optimal substrates sequences can be determined for both sides of the cleavage site.

The strategy presented herein monitors the entire substrate space of, for example, an eight amino acid sequence (˜25,600,000,000), in two discrete experiments employing a limited number of wells. Other strategies used to provide substrate specificity information such as substrate phage and bead-based methods are selection methods that identify only an optimal sequence. All additional information is lost. While potent substrates can be identified, the entirety of the information is needed to directly design selective substrates. The present invention provides this and more as will be evident upon reading the entire disclosure. For example, the assay methods presented herein provide continuous monitoring of a fluorogenic signal. With easy to control parameters such as substrate concentration and enzyme concentration, key kinetic parameters can also be determined. This is in contrast to bead-based or phage-display methods, which do not provide kinetic parameters.

For example, in bead-based strategies, without prior information, all of the queried substrate space can be represented in one construct where active beads are assayed, selected and sequenced. However, it is difficult to determine where along the amino acid chain cleavage occurred, and if there were multiple cleavage events. Accordingly, the interpretation of the information gathered becomes significantly more difficult. In addition, bead-handling and deconvolution and identification of cleavage sequences in parallel is very difficult. There are also activity profile discrepancies for the cleavage of substrates attached to a bead, and identical substrates in solution. See, e.g., Lam, K. S. & Lebl, M. (1998) Methods in Molecular Biology 87, 1-6. The present methods are performed on substrates in solutions with positional encoding with fluorogenic plate reading to overcome the above-mentioned difficulties.

Substrate phage methods are limited by the difficulties that representing all of the queried substrate space in one construct presents because there are limits to the bacterial transformation efficiencies. Therefore prior substrate specificity information is often needed to construct the library. See, e.g., Ding, L., Coombs, G. S., Strandberg, L., Navre, M., Corey, D. R. & Madison, E. L. (1995) Proceedings of the National Academy of Sciences of the United States of America 92, 7627-31; and Matthews, D. J. & Wells, J. A. (1993) Science 260, 1113-7.

In addition, using the methods provided herein, multiple copies of a positional scan can be made and stored for use in obtaining prime-side information. When non-prime specificity information is gathered, e.g., using the fluorophore-based methods, a stored positional scan library can be taken out and customized with a specific non-prime sequence. Cleavage and assay techniques presented herein provides a extremely flexible and fast technology platform for profiling enzyme substrates.

Typically, a non-prime optimal sequence is identified by methods well known to those of skill in the art or by using the high purity libraries described above. The non-prime sequence information is then used to bias the composition of a donor-quencher construct in a positional scanning format to obtain prime-side substrate specificity information. In essence, the non-prime information gathered in a first profiling experiment is used to fix the catalytic register of a second library, e.g., a donor-quencher library, thus reducing the total number of variable library positions. As a consequence, the complexity of the donor-quencher library is vastly reduced allowing for straightforward interpretation of prime side profiling results. In this manner, a complete substrate profile is obtained. The complete substrate profile conveniently provides optimal substrate compositions, e.g., amino acid or non-peptide sequences, for both sides of an enzyme cleavage site, as well as kinetic data.

In brief, the methods typically comprise profiling a substrate library, e.g., a fluorophore-based substrate library, using techniques known in the art or those presented above, to reveal an optimal amino acid or non-peptide molecule sequence for the nonprime positions of a substrate of interest or a first library of substrates. Next, a second library is prepared, a prime side scan library. Typically, a library for a prime scan, a library for probing prime side substrate sequence specificity, is prepared using a donor-acceptor pair and the optimal non-prime sequences obtained in the previous step. The prime side scan library is then incubated with the enzyme of interest and monitored to determine one or more optimal prime substrate sequence.

For example, a typical method comprises providing a library of putative protease substrates, each of which comprises a putative protease recognition site and incubating the library with the protease. The substrate profile is obtained by monitoring cleavage of the putative protease substrates by the protease, thereby providing the substrate profile for the protease.

The putative protease substrate library comprises a plurality of putative substrates, with putative, e.g., proposed, supposed, or potential recognition sites. The recognition sites typically comprise one or more non-prime positions and one or more prime positions, each of which positions is occupied by a substrate moiety, wherein the prime and non-prime positions flank a putative protease cleavage site. The substrate moieties typically comprise amino acids, peptides, non-peptides, organic molecules, and the like, Those in the non-prime positions are typically preselected to encourage or allow cleavage of the substrate at the putative protease cleavage site by the protease; and those that occupy one or more of the prime positions vary among different members of the library of protease substrates. FIG. 2 illustrates one plan for obtaining a plurality of different recognition sites, and other schemes are also available.

For detection purposes a fluorescence resonance energy transfer pair can be used. For example, a donor and acceptor pair can be attached to the protease substrate on either side of the putative cleavage site. Once the substrate is cleaved, the donor and acceptor are no longer held in close proximity and a change in fluorescence is observed.

Constructing Non-Prime Position Substrates

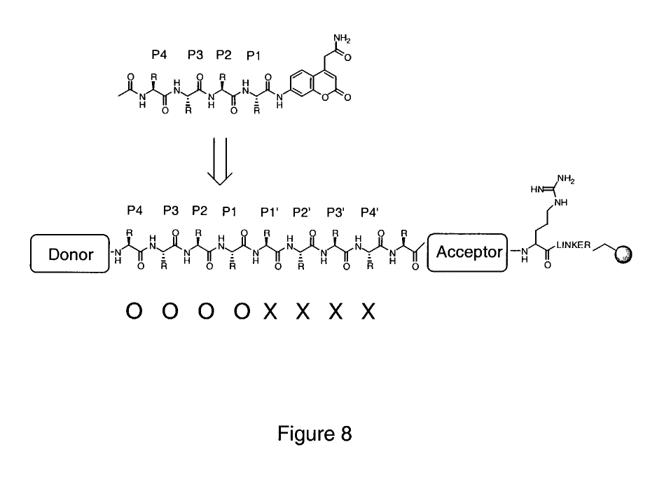

Typically, to obtain a complete substrate profile for an enzyme, such as a protease, a non-prime scan and a prime scan are performed. “Non-prime” and “prime” refer to the sides of an enzyme cleavage site. Nomenclature for the substrate amino acid preference is Pn, Pn-1, . . . P2, P1, P1′, P2′, . . . , Pm-1′, Pm′. A protease typically cleaves a substrate between P1 and P1′. The substrates typically comprise a sequence of residues, e.g., amino acids or non-peptidic molecules. Those residues on one side of the cleavage site are herein referred to as non-prime, e.g., the amino terminus side of a protein substrate, and the other side is referred to as prime. See, e.g., FIG. 8. A “non-prime scan” refers to the scanning library used to determine an optimal substrate sequence for the non-prime side of the cleavage site and/or the results of an analysis of that library. A “prime side scan” refers to the opposite side of the cleavage site, either the library used to probe those positions or the results of such a probe.

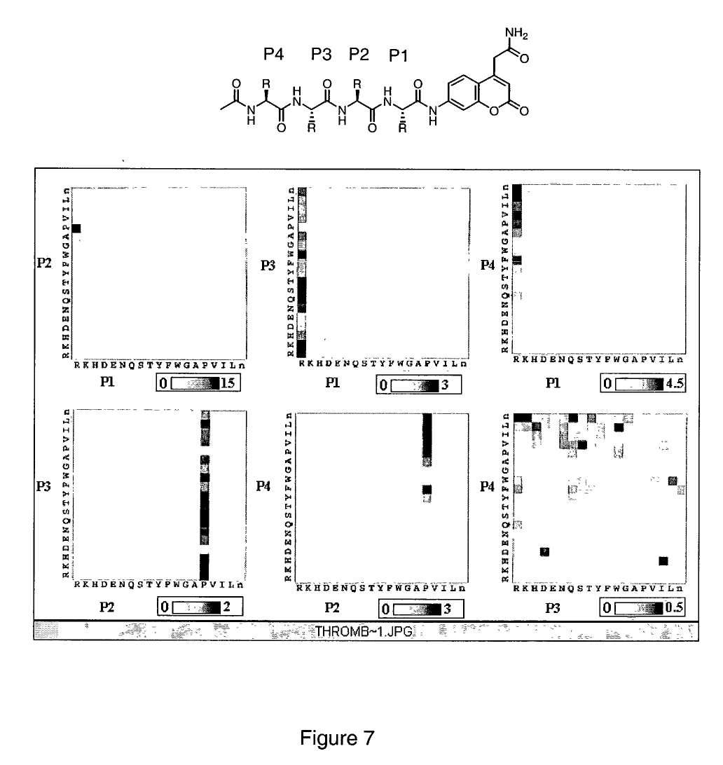

Non-prime scanning libraries are known to those of skill in the art. See, e.g., Harris et al. (2000) Proc. Nat'l. Acad. Sci. USA 97, 7754-7759. For example a coumarin-based library is used to determine an optimal amino acid sequence for the nonprime sequence for thrombin substrates. See, e.g., FIG. 7. FIG. 7 illustrates an example substrate for a non-prime scan library. The substrate shown comprises a coumarin compound and four substrate moieties or residues, e.g., P1, P2, P3, and P4.

Libraries of substrates are typically created using techniques well known to those of skill in the art or the methods provided herein for producing high purity libraries and/or non-peptide libraries. A library plan similar to that provided in FIG. 2 is optionally used. For example, a sub-library is provided wherein one of the four positions, P1-P4, is fixed while the others are varied. Another sub-library can have another of P1-P4 fixed, while the other positions are varied, and so on. In addition, libraries comprising two fixed residues are also optionally created. These libraries are typically incubated with the enzyme of interest and the released coumarin compound is detected, e.g., fluorescently, to provide an analysis of the optimal residues for positions P1-P4.

FIG. 7 provides data obtained from incubating a non-prime scan library of coumarin-based substrates with thrombin. When thrombin acts on a substrate, the substrate is cleaved between P1 and the coumarin moiety, thereby releasing the fluorogenic coumarin moiety, which is detected. As shown in FIG. 7, arginine is an optimal P1 residue and proline is an optimal P2 residue. P3 is variable and P4 favors aliphatic and aromatic residues.

To provide a complete substrate profile of an enzyme, a non-prime side scan is typically performed to obtain one or more preferred and/or optimal non-prime substrate sequence. Such an analysis is referred to herein as “positional scanning.” See also, Rano et al. (1997) Chem. Biol. 4, 149-155.

In the manner described above, an “optimal non-prime substrate moiety” is determined. This is the optimal or preferred sequence of residues for an enzyme of interest to cleave a substrate. In the present invention, the optimal non-prime substrate moiety is typically used to create a second library, which is used to probe the prime side substrate specificity. In this way, the methods provided herein provide a more complete profile of substrate specificity than those methods presently known in the art.

Constructing Prime Position Substrates

To further probe substrate specificity of an enzyme by providing prime as well as non-prime specificity information, a second library is typically created, e.g., in addition to the non-prime side substrate library described above that is used to probe nonprime substrate specificity and from which a non-prime sequence is preselected. The prime position substrates and libraries provided herein take advantage of information obtained from a non-prime scan, e.g., to provide preselected non-prime substrate sequences.

A prime side position library is typically constructed using a donor and acceptor detection pair, e.g., a FRET pair, and a preselected non-prime substrate sequence. Donor moieties and acceptor moieties in the present invention typically comprise fluorescence resonance energy transfer pairs. A typical donor of the invention absorbs light at one wavelength and emits at another wavelength, typically a higher wavelength. The acceptor moiety of the invention typically absorbs at the wavelength of either the absorption or emission wavelength of the donor moiety. For example, the acceptor is used as a quencher for the donor moiety. However, the acceptor typically only quenches the absorption or emission of the donor when the two are in proximity, either in high concentrations or when tethered to each other, e.g., chemically bonded as in the example shown in FIG. 8. The donor-acceptor pairs are then used to detect protease cleavage of the substrates of the libraries in the present invention, e.g., when cleavage occurs, the acceptor no longer quenches the signal of the donor, as explained in more detail below.

One or more prime position substrate moiety is typically coupled to an acceptor moiety. The prime substrate moieties typically comprise amino acids, peptides, non-peptide molecules, organic molecules, and the like. In a typical library, about four substrate moieties are coupled to the acceptor, e.g., P1′, P2′, P3′, and P4′. However, the number of substrate moieties coupled to the acceptor is optionally varied, e.g., from about 1 to about 15, but is more typically, about 2 to about 6, and most typically four. Typically, the substrate moieties are coupled to an acceptor using standard peptide synthesis techniques, e.g., Fmoc synthesis.

After the prime side positional substrate is coupled to the acceptor, a preselected non-prime substrate, e.g., an optimal or preferred non-prime sequence that has been identified as described above, is coupled to the prime position substrate.

After a preselected non-prime positional substrate sequence has been added to the prime position substrate/acceptor moiety, a donor is coupled to the preselected nonprime substrate. The donor typically comprises one member of a FRET pair as described above, e.g., aminobenzoic acid, 7-methoxy-4-carbamoylmethyl coumarin, 7dimethylamino-4-carbamoylmethyl coumarin, or the like. In alternate embodiments, the donor moiety is coupled to the prime side substrate and the acceptor moiety is coupled to the preselected non-prime substrate.

These libraries are optionally made using solid phase peptide synthesis methods as described, e.g., Harris et al. (2000) PNAS 97, 7754-7759, or they are optionally constructed using the methods provided above, e.g., to produce high purity libraries using novel coumarin and linker groups that allow protecting groups to be removed from the substrate and washed away prior to cleavage of the substrate from the support. In addition, the non-peptide techniques described above are also optionally used to create prime position substrate libraries, e.g., in combination with non-prime position libraries, e.g., preselected non-prime position libraries. For example, the substrate moieties, e.g., P1′, P2′, P3′, P4′, and the like, are optionally non-peptide molecules, e.g., instead of amino acids.

For example, a substrate for use in a prime position library is typically made by coupling an acceptor moiety, e.g., a FRET acceptor, to a solid support, e.g., a polystyrene or polypropylene resin. Acceptors of the invention include, but are not limited to, nitro-tyrosine, dinitrophenol-lysine, dabsyl-lysine, and the like. Other solid supports available include, but are not limited to, polyacrylamide, polyethylene glycol, and the like. In some embodiments, the acceptor is coupled to the solid support via a linker, e.g., an arginine linker as shown in FIG. 8. Rink linkers, glycol linkers, or any other linker moiety typically used in peptide synthesis protocols are also optionally used.

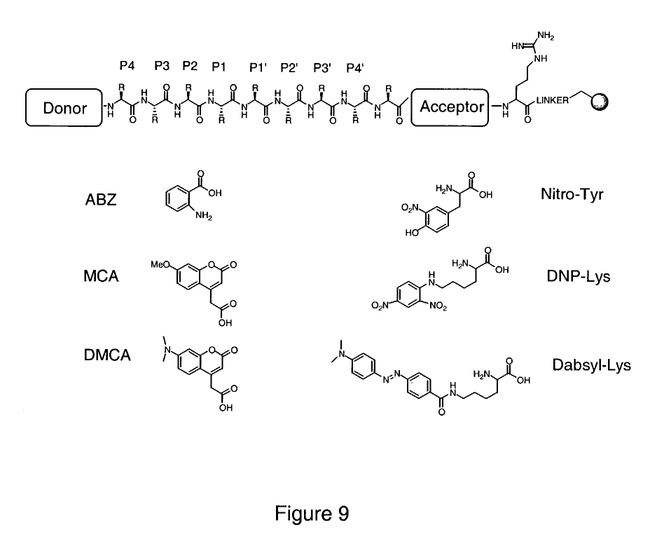

FIG. 8 provides an example dual positional scan substrate, e.g., a positional scan substrate capable of probing both prime and non-prime substrate sequences. FIG. 8 illustrates the use of a preselected non-prime position substrate for use with a prime position substrate. An acceptor is coupled to a solid support, e.g., a PEG particle, via an arginine linker. The prime side substrate is coupled to the acceptor and a preselected, e.g. preferred, non-prime position substrate sequence is coupled to the prime side substrate. For example, a preferred non-prime sequence for a thrombin substrate comprises P1-arginine, P2-Proline, P3-variable, and P4-an aliphatic or aromatic residue. A donor is then coupled to the preselected non-prime substrate. Example donor/acceptor pairs include, but are not limited to, aminobenzoic acid and nitro-tyrosine, the other donor/acceptor pairs provided in FIG. 9, and others that are well known to those of skill in the art. Using a library of substrates like the one shown in FIG. 8 provides a library tailored to a specific protease, e.g., thrombin. By coupling the preselected non-prime substrate directly to the prime side substrate, the cleavage site is set.

Once one or more non-prime sequences, e.g., optimal or preferred sequences, are selected or identified, e.g., using standard native sequences, or performing a positional non-prime scan as described above, a library of substrates is constructed, e.g., as depicted in the plan of FIG. 2. Alternate plans are also available. For example, libraries can be constructed using 1, 2, 3, or more fixed positions. For example, substrates are optionally created in which more than four positions are provided and profiled on each side of the cleavage site. More than one preselected non-prime sequence is optionally used to create multiple libraries to scan the prime side of the cleavage site, e.g., to obtain more complete profiling results. Once the libraries are created, they are analyzed as described below to determine optimal prime side substrate moieties.

Determination of an optimal or preferred prime position substrate

A library of substrates, e.g., as described above, is typically incubated with an enzyme of interest, to determine substrate specificity. For example, a library created with a non-prime substrate moiety tailored to thrombin substrates is used to create a library to identify prime side thrombin substrate sequences. Therefore, such a library would be incubated with thrombin. The enzyme is added to the library, which has typically been released from the solid support. For example, for a library comprising 600 microwells with multiple sequences in each, enzyme is added to each of the 60 wells.

Fluorescence is typically detected continuously, at multiple time points in the course of the enzymatic reaction, or at a single time point at or near the end of the reaction. By continually monitoring the fluorescence in each well of the library, kinetic data is also optionally obtained. The detection is used to monitor which wells, e.g., which substrates are cleaved by the enzyme. Using a library of substrates as shown in FIG. 8, the concept of fluorescence resonance energy transfer is used to detect when the donor is cleaved from the acceptor.

Fluorescence resonance energy transfer (FRET) is a distance dependent excited state interaction in which emission of one fluorophore is coupled to the excitation of another fluorophore which is in proximity, e.g. close enough for an observable change in emissions to occur. In the present application, the donor and acceptor interact when in proximity, e.g., due to FRET. Typically, the donor and acceptor are located on opposite sides of the cleavage site. When a protease is incubated with the libraries of the present invention, e.g., the prime side scan libraries, cleavage occurs in between P1 and P1′, therefore separating the donor from the acceptor. When the two are in proximity, e.g., in an intact substrate, the acceptor quenches the donor and little or no signal is observed. When cleavage occurs, the donor and the acceptor are separated physically and the acceptor no longer quenches the donor signal. The donor then emits a signal that is observed by a detector. Typically, in the present invention, detection is monitored continuously, e.g., at multiple time points. The data obtained in this manner is then optionally used to provide kinetic information regarding the enzyme activity.

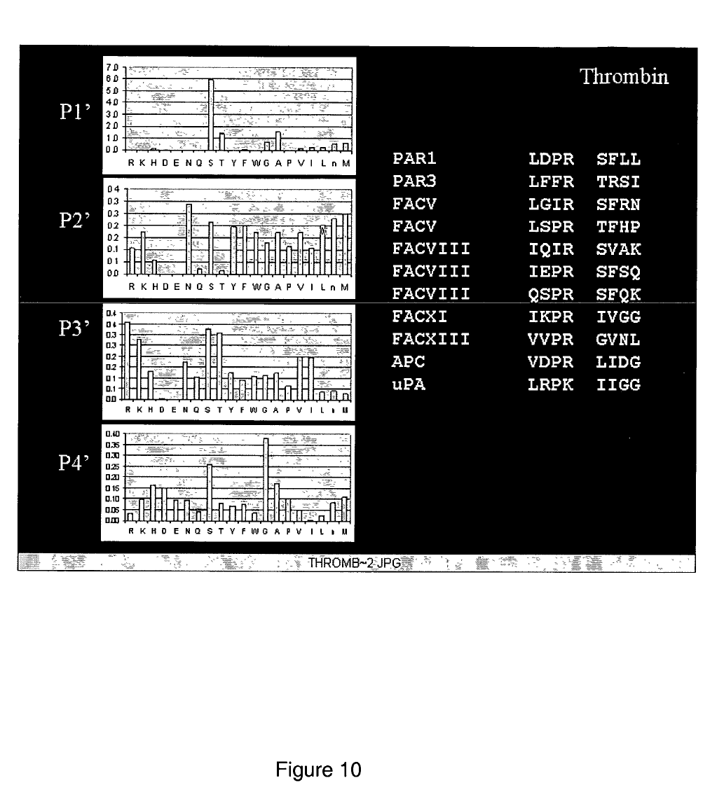

FIG. 10 provides data from a thrombin substrate profile obtained using the methods described herein. Optimal or preferred substrate moieties are provided for P1′, P2′, P3′, and P4′ as shown in the graphs on the left of FIG. 10. The first column on the right side of FIG. 10 lists known biological substrates for thrombin and the second and third columns provide known non-prime (second column) and prime cleavage (third column) sites for the listed substrates. As seen by comparing the graph to the lists, the profiles provide accurate information regarding substrate specificity. Therefore, the present invention provides the ability to rapidly obtain complete substrate profiles, e.g., of both sides of a cleavage site.

In addition, the prime and non-prime information can be used to search genomic databases for similar cleavage sites in proteins and provide possible macromolecular substrates that are key to the biological function of the protease of interest. The prime side information is optionally used to construct nucleophilic compounds that sit in the prime binding pocket and intercept the O-acyl intermediates formed during cleavage, e.g., of macromolecular substrates. These molecules are optionally used to identify novel macromolecular substrates of a specific protease, e.g., in complex biological samples.

The prime and non-prime information is also optionally used to design more selective and potent substrates, e.g., for use as therapeutic agents or biological tools. Multiple fluorogenic compounds can be employed with the determined amino acid specificity sequence to increase the sensitivity and efficacy of these substrates for a particular system.

Furthermore, substrates of the present invention are very valuable as diagnostics for the identification of protease activity in complex biological samples and for screening efforts to identify protease inhibitors. The overall strategy when applied to an entire class of proteases provides panning information that allows for the generation of specific substrates and inhibitors in the context of an entire protease class.

The non-prime and prime specificity information can be employed to bias bead-based and phage display methods, to design cleavage sites in fusion proteins or other protein constructs, and to design prodrugs in which the protease target releases an active drug.

In another embodiment, the present invention provides databases constructed using the above substrate profile information. These data bases are optionally used in the applications described above, e.g., to design improved protease substrates, for use in identifying proteases inhibitors, for use in characterizing proteases for which substrates were previously unknown or incompletely characterized, and the like.

A database of the invention typically comprises records for members, e.g., each member, of a library of putative protease substrates, e.g., the libraries described herein. Each record typically comprises information regarding the identity of a substrate moiety or group of substrate moieties, e.g., amino acids, peptides, or non-peptides, that occupy each of one or more prime and non-prime positions of a particular putative protease substrate. Data from assays used to determine the ability of the proteases to cleave the putative protease substrate is also included in the database, as well as kinetic data obtained from the assay, e.g., by detecting at multiple time points in the course of the reaction.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method of preparing one or more fluorophore-containing enzyme substrates, the method comprising:
(a) coupling one or more fluorogenic compounds to a solid support via an ammonia-cleavable linker, resulting in one or more support-bound fluorogenic compounds;
(b) coupling one or more substrate moieties to the support-bound fluorogenic compound to form a fluorophore-containing enzyme substrate;
(c) exposing the support-bound fluorogenic compound to ammonia, thereby releasing the fluorogenic compound from the support, resulting in a soluble fluorophore-containing enzyme substrate.

2. The method of claim 1, wherein the fluorogenic compound comprises a coumarin compound.

3. The method of claim 2, wherein the coumarin compound comprises 7amino-4-carbamoylmethylcoumarin, 7-amino-4-methylcoumarin, or 7-amino-3carbomoylmethyl-4-methylcoumarin.

4. The method of claim 1, wherein the fluorogenic compound comprises a protecting group.

5. The method of claim 4, wherein the protecting group is base-labile.

6. The method of claim 5, wherein the protecting group is Fmoc.

7. The method of claim 4, further comprising removing the protecting group prior to step (b).

8. The method of claim 1, wherein the solid support comprises a polymer.

9. The method of claim 8, wherein the solid support comprises polyethylene glycol, polyethylene, polystyrene, or polyacrylamide.

10. The method of claim 1, wherein the linker moiety is stable to Fmoc deprotection.

11. The method of claim 10, wherein the linker moiety comprises a glycol linker.

12. The method of claim 1, wherein the substrate moieties are amino acids.

13. The method of claim 12, wherein the amino acids comprise a protecting group which is removed prior to coupling an additional amino acid.

14. The method of claim 13, wherein the protecting group is not ammonia-labile.

15. The method of claim 1, wherein (b) comprises performing Fmoc-based peptide synthesis.

16. The method of claim 15, wherein performing Fmoc-based peptide synthesis comprises:
(i) coupling a first Fmoc-protected amino acid to the support bound fluorogenic compound, resulting in a bound Fmoc-protected amino acid;
(ii) deprotecting the bound Fmoc-protected amino acid, resulting in a first bound amino acid;
repeating steps (i) and (ii) to add a desired number of additional bound amino acids.

17. The method of claim 16, wherein one or more of the amino acids comprises a side chain protecting group and the method further comprises:
(iv) removing one or more side chain protecting groups from the bound amino acids.

18. The method of claim 17, wherein (iv) comprises performing an acid deprotection, which acid deprotection does not release the support bound fluorophore-containing substrate from the support.

19. The method of claim 17, wherein the side chain protecting group is an acid-labile protecting group.

20. The method of claim 1, further comprising deprotecting the substrate moiety after step (b) and prior to step (c).

21. The method of claim 1, wherein the ammonia comprises gaseous ammonia.

22. The method of claim 1, wherein the fluorophore-containing substrate is a protease substrate.

23. The method of claim 1, wherein the fluorophore-containing substrate comprises one or more peptide or protein.

24. The method of claim 1, wherein the one or more fluorophore-containing substrate comprises a library of fluorophore-containing substrates.

25. The method of claim 24, wherein the library comprises a high purity library.

26. The method of claim 24, wherein the library comprises a positional-scanning library.

27. The method of claim 26, wherein the positional scanning library comprises a protease substrate positional-scanning library.

28. The method of claim 24, wherein the library is substantially free of protecting group derived side products.

29. The method of claim 28, wherein the library is substantially free of other side products.

30. The method of claim 24, wherein the library comprises greater than 50 members.

31. The method of claim 30, wherein the library comprises greater than 100 members.

32. The method of claim 31, wherein the library comprises greater than 1,000 members.

33. A fluorophore-containing enzyme substrate that comprises an ammonia-labile linker.

34. The fluorophore-containing enzyme substrate of claim 33, wherein the linker comprises a glycol linker or a benzylalcohol linker.

35. The fluorophore-containing enzyme substrate of claim 33, further comprising one or more amino acid or one or more non-peptide moiety.

36. The fluorophore-containing enzyme substrate of claim 33, wherein the enzyme substrate comprises a protease substrate.

37. The fluorophore-containing enzyme substrate of claim 33, wherein the fluorophore-containing enzyme substrate is substantially free of protecting groups.

38. A method of obtaining a substrate profile for a protease, the method comprising:
(a) providing a library of putative protease substrates, each of which comprises a putative protease recognition site, wherein:
(i) the putative protease recognition site comprises one or more non-prime positions and one or more prime positions, each of which positions is occupied by a substrate moiety, wherein the prime and non-prime positions flank a putative protease cleavage site;
(ii) the substrate moieties that occupy one or more of the nonprime positions are preselected to allow cleavage of the substrate at the putative protease cleavage site by the protease; and
(iii) the substrate moieties that occupy one or more of the prime positions vary among different members of the library of protease substrates;
(b) incubating the library in the presence of the protease; and
(c) monitoring cleavage of the putative protease substrates by the protease, thereby providing the substrate profile for the protease.

39. The method of claim 38, wherein cleavage of the protease substrate compounds is detected by fluorescence resonance energy transfer.

40. The method of claim 39, wherein a fluorescence donor moiety and a fluorescence acceptor moiety are attached to the protease substrate compound on opposite sides of the putative protease cleavage site.

41. The method of claim 38, wherein the substrate moieties that occupy one or more of the prime positions are selected so as to comprise a positional scanning combinatorial library.

42. The method of claim 38, wherein the substrate moieties that occupy one or more of the non-prime positions are preselected by:
(a) providing a first library that comprises one or more putative protease substrates, each of which comprises one or more non-prime positions, each of which positions is occupied by a substrate moiety;
(b) incubating the library in the presence of the protease; and
(c) identifying library members that are cleaved by the protease, thereby identifying substrate moieties that, when present in a particular non-prime position, allow cleavage of the substrate by the protease.

43. The method of claim 42, wherein the putative protease substrates comprise a fluorogenic compound.

44. The method of claim 43, wherein cleavage of the members of the first library is determined by detecting a shift in the excitation and/or emission maxima of the fluorogenic compound, which shift results from release of the fluorogenic compound from the putative protease substrate by the protease.

45. The method of claim 43, wherein the method further comprises determining one or more kinetic constants for release of the fluorogenic compound.

46. The method of claim 42, wherein the first library comprises fluorophore-containing substrates which are synthesized by a method that comprises:
a) coupling one or more fluorogenic compounds to a solid support via an ammonia-cleavable linker, resulting in one or more support-bound fluorogenic compounds;
b) coupling one or more substrate moieties to the support-bound fluorogenic compound to form fluorophore-containing substrates; and
c) exposing the support-bound fluorophore-containing substrates to ammonia, thereby releasing the fluorophore-containing substrates from the support, resulting in a fluorophore-containing enzyme substrate.

47. The method of claim 38, wherein the members of the library are each attached to solid supports.

48. The method of claim 38, wherein the putative protease recognition site comprises two or more non-prime and two or more prime positions.

49. The method of claim 48, wherein the putative protease recognition site comprises four non-prime and four prime positions.

50. A database of substrate profile information for a protease, wherein the database comprises records for members of a library of putative protease substrates, each record comprising:
(a) information as to the identity of a substrate moiety that occupies each of one or more prime and non-prime positions of the particular putative protease substrate;
(b) data from assays to determine the ability of the protease to cleave the particular putative protease substrate.

51. The database of claim 50, wherein the assay data comprises kinetic data.

52. The database of claim 50, wherein the assay data is obtained by a method comprising:
(a) providing a library of putative protease substrates, each of which comprises a putative protease recognition site, wherein:
(i) the putative protease recognition site comprises one or more non-prime positions and one or more prime positions, each of which positions is occupied by a substrate moiety, wherein the prime and non-prime positions flank a putative protease cleavage site;
(ii) the substrate moieties that occupy one or more of the nonprime positions are preselected to allow cleavage of the substrate at the putative protease cleavage site by the protease; and
(iii) the substrate moieties that occupy one or more of the prime positions vary among different members of the library of protease substrates;
(b) incubating the library in the presence of the protease; and
(c) monitoring cleavage of the putative protease substrates by the protease.

53. A method of obtaining a substrate profile for a protease, the method comprising:
(a) providing a first library comprising a plurality of putative protease substrates that each comprise a fluorogenic compound and one or more non-prime positions, each of which is occupied by a substrate moiety;
(b) analyzing the first library to identify substrate moieties at one or more non-prime positions that result in cleavage of the putative protease substrate by a protease;
(c) constructing a second library, wherein constructing the second library comprises:
(i) coupling to a first member of a fluorescence resonance energy transfer pair a substrate moiety in each of one or more prime positions;
(ii) coupling to a second member of the fluorescence resonance energy transfer pair a substrate moiety at one or more non-prime positions that were determined in step b) to result in cleavage of the substrate by a protease; and,
(iii) linking the compounds of (i) and (ii) together to form the second library;
(d) incubating the second library with the enzyme; and
(e) monitoring the fluorescence resonance energy transfer to identify one or more optimal prime substrate moiety, thereby providing the substrate profile for the enzyme.

54. The method of claim 53, wherein the protease comprises a serine protease, a threonine protease, a metalloprotease, a cysteine protease, or an aspartyl protease.

55. The method of claim 53, wherein the protease comprises thrombin, caspase, plasmin, factor Xa, tissue plasminogen activator, trypsin, chymotrypsin, elastase, papain, or cruzain.

56. The method of claim 53, wherein the fluorescent resonance energy pair comprises amino benzoic acid and nitro-tyrosine; 7-methoxy-4carbomoylmethylcoumarin and dinitrophenol-lysine, or 7-dimethylamino-4carbomoylmethylcoumarin and Dabsyl-Lysine.

57. A library of putative protease substrates, each of which comprises a putative protease recognition site, wherein:
(i) the putative protease recognition site comprises one or more nonprime positions and one or more prime positions, each of which positions is occupied by a substrate moiety, wherein the prime and non-prime positions flank a putative protease cleavage site;
(ii) the substrate moieties that occupy one or more of the non-prime positions are preselected to allow cleavage of the substrate at the putative protease cleavage site by the protease; and
(iii) the substrate moieties that occupy one or more of the prime positions vary among different members of the library of protease substrates;

58. The library of claim 57, wherein the putative protease substrates are substantially free of protecting groups.

59. A method of identifying one or more non-peptide substrates, the method comprising:
(a) providing a support bound fluorogenic compound;
(b) coupling one or more amino acids to the support bound fluorogenic compound;
(c) coupling one or more non-peptide molecules to the amino acid to form a putative non-peptide protease substrate; and,
(d) contacting the putative non-peptide protease substrate with a protease to determine whether the protease cleaves the putative substrate.

60. The method of claim 59, wherein the amino acid comprises aspartic acid.

61. The method of claim 59, step (c) comprising performing solid phase synthesis.

62. The method of claim 59, wherein step (c) comprises forming a heterocycle moiety on the amino acid.

63. The method of claim 59, wherein step (c) comprises benzodiazepine solid phase synthesis.

64. The method of claim 59, wherein the putative non-peptide protease substrate is released from the support prior to contacting the substrate with the protease.

65. The method of claim 59, wherein the fluorogenic compound comprises a coumarin compound.

66. A method of identifying one or more non-peptide substrates for a protease, the method comprising:
(a) providing a putative protease substrate that comprises: a fluorogenic compound, an amino acid attached to the fluorogenic compound, and one or more non-peptide molecules attached to the amino acid;
(b) contacting the putative protease substrate with a protease;
(c) determining whether the protease cleaves the putative protease substrate by detecting a shift in the excitation and/or emission maxima of the fluorogenic compound, which shift results from cleavage of the fluorogenic compound from the amino acid.

67. The method of claim 66, wherein the fluorogenic compound is a coumarin compound.

68. The method of claim 67, wherein the coumarin compound is selected from the group consisting of: 7 amino-3-carbomoylmethyl-4-methylcoumarin; 7-amino-4carbamoylmethylcoumarin, and 7-amino-4-methylcoumarin.

69. A library of non-peptide substrates made by the method of claim 59.

70. A library of coumarin based non-peptidic protease substrates.

71. The library of claim 70, wherein the protease comprises a serine protease, a threonine protease, a metalloprotease, a cysteine protease, or an aspartyl protease.

72. The library of claim 70, wherein the protease comprises thrombin, caspase, plasmin, factor Xa, tissue plasminogen activator, trypsin, chymotrypsin, elastase, papain, or cruzain.
